(19) United States
(12) Patent Application Publication
(10) Pub. No.: US 2025/0057433 A1
(43) Pub. Date: Feb. 20, 2025

(54) INTRACRANIAL PRESSURE SENSING GUIDEWIRE

(71) Applicant: KONINKLIJKE PHILIPS N.V., Eindhoven (NL)

(72) Inventor: Meghan B. MORRILL, COLORADO SPRINGS, CO (US)

(21) Appl. No.: 18/770,801

(22) Filed: Jul. 12, 2024

Related U.S. Application Data
(60) Provisional application No. 63/533,380, filed on Aug. 18, 2023.

(51) Int. Cl. A61B 5/03; A61B 5/00

(52) U.S. Cl. CPC A61B 5/031; A61B 5/6851; A61B 5/7264; A61B 5/7405; A61B 5/742; A61B 5/746; A61B 2560/0223; A61B 2560/0462; A61B 2562/0247; A61B 2562/227

(57) ABSTRACT

An apparatus includes an intracranial guidewire, with a flexible elongate member configured for insertion into a subarachnoid space through a skull of a patient, and a pressure sensor disposed at a distal portion of the flexible elongate member and configured to measure a pressure of cerebrospinal fluid inside of the subarachnoid space.

### TECHNICAL FIELD

The subject matter described herein relates to devices, methods, and systems for intracranially measuring pressure of cerebrospinal fluid. This intracranial pressure sensing guidewire has particular but not exclusive utility for diagnosis and treatment of head trauma.

### BACKGROUND

Traumatic brain injuries (TBI) occur when a sudden trauma causes damage to the brain such as when a patient's head violently hits something, or an object pierces the skull and brain tissue. This can occur for example in falls, car accidents, sports injuries, explosions, etc. In the United States alone, approximately 1.5 million people sustain a TBI with 50,000 deaths and ˜90,000 people left with long-term disabilities each year.

Symptoms of a TBI can range from mild to severe, and range from loss of memory, loss of consciousness, vertigo, slurred speech, seizures, and even coma or death. The type of symptoms depends on the severity of the impact and where the trauma occurs within the cranial cavity. With a TBI comes the increased risk for a rise in intracranial pressure (ICP) which is critical.

With a TBI, the flow of cerebral spinal fluid (CSF), which provides a mechanical barrier against shock, can be restricted, and can cause the brain to swell. One of the most critical impacts can be the rise of intracranial pressure (ICP), wherein the brain swells against the confinement of the skull. The mechanical properties of brain tissue make it incompressible, so that as the lobes swell, CSF and blood flow decrease, leading to edema, lesions, and tissue injury. Treatment to relieve the increase in cranial pressure can include medications (diuretics, coma-inducing drugs), surgeries to remove resulting lesions and hematomas, removal of skull bone to allow the brain to swell with no resistance, or directly draining any built up below the brain in the spinal cord.

Concern for rising intracranial pressure is dependent on several clinical indications for a TBI, including without limitation: a Glasgow Coma Scale (GCS) value lower than 8, abnormal computed tomogram (CT) results, a patient older than 40 years, a low systolic blood pressure, patient injuries with large bifrontal contusions, a non-reliable neurology exam. If high ICP is suspected for these or other reasons, a clinician (e.g., a paramedic) may create burr holes in the skull (e.g., with a specialized drill) to relieve the pressure. Fluid leakage from the burr hole may be a crude indicator of high ICP. However, there is no specific clinical feature or symptom that indicates an increase, or a test such as MRI or CT that can fully detect ICP. Roughly 50% of patients that develop high ICP show a clear head CT scan. It is thus to be appreciated that such commonly used ICP estimation methods have numerous drawbacks, including poor precision, low confidence, and a high incidence of error. Accordingly, a need exists for improved diagnostic methods that can be used outside a hospital setting.

The information included in this Background section of the specification, including any references cited herein and any description or discussion thereof, is included for technical reference purposes only and is not to be regarded as subject matter by which the scope of the disclosure is to be bound.

### SUMMARY

Disclosed is an intracranial pressure sensing guidewire configured measure the pressure of cerebrospinal fluid (CSF) and a display monitor that connects directly to the pressure-sensing guidewire to display pressure readings and to provide user feedback such as threshold alarms. In some cases, an intracranial pressure sensing system may also include other components such as an access drill and channel plug. The intracranial pressure sensing guidewire has particular, but not exclusive, utility for emergency use under field condition (e.g., in accident sites, ambulances, battlefields, etc.).

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes an intracranial guidewire that may include: a flexible elongate member configured for insertion into a subarachnoid space through a skull of a patient, and a pressure sensor disposed at a distal portion of the flexible elongate member and configured to measure a pressure of cerebrospinal fluid inside of the subarachnoid space. Other embodiments of this aspect include corresponding computer systems, apparatus, and computer programs recorded on one or more computer storage devices, each configured to perform the actions of the methods.

Implementations may include one or more of the following features. In some embodiments, the flexible elongate member may include a core wire configured to provide structural support for the intracranial guidewire. In some embodiments, the flexible elongate member does not include a lumen. In some embodiments, a length of the flexible elongate member is between 30 cm and 90 cm. In some embodiments, the apparatus may include: a plurality of electrical conductors extending along a length of the flexible elongate member and configured to carry electrical signals associated with the pressure sensor; and a connection portion disposed at a proximal portion of the flexible elongate member and in electrical communication with the pressure sensor via the plurality of electrical conductors. Implementations of the described techniques may include hardware, a method or process, or computer software on a computer-accessible medium.

One general aspect includes a system for measuring intracranial pressure, including an intracranial guidewire configured to be inserted through an opening in a skull of a patient, where the intracranial guidewire may include: a pressure sensor disposed at a distal portion of the intracranial guidewire and configured to generate signals representative of a pressure of cerebrospinal fluid inside the skull; and a connection portion disposed at a proximal portion of the intracranial guidewire and in communication with the pressure sensor; and a display monitor that may include: a housing; a connection port coupled to the housing, where the connection port is configured to receive the proximal portion of the intracranial guidewire and establish communication with the pressure sensor via the connection portion; a processor circuit disposed inside the housing and configured to receive the signals generated by the pressure sensor via the connection port and connection portion of the intracranial guidewire; and a display coupled to the housing and in communication with the processor circuit, where the display is configured to output a visual representation of the pressure of the cerebrospinal fluid based on the signals generated by the pressure sensor. Other embodiments of this aspect include corresponding computer systems, apparatus, and computer programs recorded on one or more computer storage devices, each configured to perform the actions of the methods.

Implementations may include one or more of the following features. In some embodiments, the display monitor further may include: a power supply disposed inside the housing and configured to provide power to at least one of the processor circuit, the display, or the connection port. In some embodiments, the display monitor further may include a user control coupled to the housing and in communication with the processor circuit, where an actuation of the user control by a user is configured to perform at least one of: provide power to the pressure sensor; calibrate the pressure sensor; or trigger the display of the visual representation of the pressure of the cerebrospinal fluid. In some embodiments, the opening may include the burr hole. In some embodiments, the channel plug may include a lumen extending completely through the channel plug and configured to receive the intracranial guidewire during the insertion of the intracranial guidewire through the opening. In some embodiments, the visual representation of the pressure of the cerebrospinal fluid may include a numerical value. In some embodiments, the processor is configured to: determine the pressure of the cerebrospinal fluid based on the signals generated by the pressure sensor; compare the pressure of the cerebrospinal fluid to a threshold value; determine if the pressure of the cerebrospinal fluid exceeds the threshold value based on the comparison; and activate a threshold alarm in response to the determination that the pressure of the cerebrospinal fluid exceeds the threshold value. In some embodiments, the display monitor further may include a speaker disposed inside the housing and in communication with the processor circuit, where the threshold alarm may include an audible alarm output by the speaker. In some embodiments, the display monitor further may include an indicator light coupled to the housing and in communication with the processor circuit, where the threshold alarm may include activation of the indicator light. Implementations of the described techniques may include hardware, a method or process, or computer software on a computer-accessible medium.

In some aspects, the access drill is configured to drill through a scalp, skull, periosteal dura mater, meningeal dura mater, and arachnoid mater of the cranium, and thus into a subarachnoid space of the cranium, without contacting a pia mater or cerebral cortex of the cranium. One general aspect includes a method for measuring intracranial pressure. The method includes: inserting a flexible elongate member into a cranium via a burr hole. The flexible elongate member includes: a pressure sensor disposed at a distal portion of the flexible elongate member and configured for measuring a pressure of intracranial cerebrospinal fluid when the flexible elongate member is positioned within the cranium, and a connection portion disposed at a proximal portion of the flexible elongate member and in electrical communication with the pressure sensor. The method also includes, with a display monitor: with a receiver portion configured to receive the connection portion, receiving the connection portion; with a power supply, providing electrical power to the receiver and a processor circuit comprising a memory; with the processor circuit, receiving the measured pressure via the receiver portion and connection portion; and with a display in communication with the processor circuit, displaying the measured pressure.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of the intracranial pressure sensing guidewire, as defined in the claims, is provided in the following written description of various embodiments of the disclosure and illustrated in the accompanying drawings.

### BRIEF DESCRIPTION OF THE DRAWINGS

Illustrative embodiments of the present disclosure will be described with reference to the accompanying drawings, of which:

FIG. 1 is a schematic, diagrammatic representation of at least a portion of a human head or cranium, in accordance with aspects of the present disclosure.

FIG. 2 is an exemplary representation of at least a portion of a human brain, in accordance with aspects of the present disclosure.

FIG. 3 is a schematic, diagrammatic view, in block diagram form, of an intracranial pressure sensing system, according to aspects of the present disclosure.

FIG. 4 is a diagrammatic cross-sectional view of an intracranial sensing guidewire that includes a pressure sensor, according to aspects of the present disclosure.

FIG. 5 is a diagrammatic cross-sectional view of an intracranial sensing guidewire that includes a pressure sensor, according to aspects of the present disclosure.

FIG. 6 is a diagrammatic cross-sectional view of an intracranial sensing guidewire that includes a pressure sensor, according to aspects of the present disclosure.

FIG. 7 is a side perspective cross-sectional view of an intracranial pressure sensing guidewire that includes a pressure sensor, according to aspects of the present disclosure.

FIG. 8 is a diagrammatic cross-sectional view of a display monitor, according to aspects of the present disclosure.

FIG. 9 is a diagrammatic cross-sectional view of a display monitor, according to aspects of the present disclosure.

FIG. 10 is a schematic, diagrammatic view, in block diagram form, of the wiring of an intracranial pressure sensing guidewire that includes a pressure sensor, according to aspects of the present disclosure.

FIG. 11 is a side perspective view of an access drill or intraosseous drill, according to aspects of the present disclosure.

FIG. 12 is a side perspective view of a channel plug, according to aspects of the present disclosure.

FIG. 13 is a top perspective view of a channel plug, according to aspects of the present disclosure.

FIG. 14 is a diagrammatic, side cross-sectional view of an intracranial pressure sensing guidewire inserted into the subarachnoid space of a human cranium, according to aspects of the present disclosure.

FIG. 15 is a diagrammatic side view of an intraluminal (e.g., intravascular) sensing system that includes an intravascular pressure-sensing device, according to aspects of the present disclosure.

FIG. 16 is a schematic, diagrammatic size comparison between an intracranial pressure-sensing guidewire and an intraluminal or intravascular pressure-sensing guidewire 2102, according to aspects of the present disclosure.

FIG. 17 is a schematic diagram of a processor circuit, according to aspects of the present disclosure.

### DETAILED DESCRIPTION

To determine definitively whether a victim of TBI is suffering from an increase in ICP, it may be necessary to measure the ICP directly. In-hospital methods to measure the pressure within the skull have been developed.

For example, lumbar CSF pressure measurement involves inserting a pressure measuring catheter into the lumbar region of the spinal column, which will measure the pressure of the CSF for 24 to 72 hours. This method is extracranial and does not require placing an instrument in the brain, but does require insertion near the spinal cord, with attendant risk of injury to the spinal nerves.

A subdural bolt involves a hole being drilled into the skull and a hollow screw then placed invasively through the membrane of the brain, until it is within the subdural space. This device can provide accurate pressure values, but is highly invasive, with requiring burr holes up to 12 mm in diameter which, when the device is removed, may need to be sealed with a metal plate. The subdural bolt is also made of a shorter, more rigid material, so that the bolt is relatively not flexible.

Epidural transducer-type sensors involve a hole drilled into the skull, where a pressure sensor is placed beneath the bone and the first brain tissue layer (dura) of the brain. As the sensor does not enter the dura mater tissue, it is less invasive than the bolt. Placement of the device does not involve penetration of the brain tissues or layers. Drawbacks of many epidural sensors is that they are not inserted directly into locations with CSF and often sit within the skull (bone) above CSF location therefore, may not measure CSF build up. Additionally, the material of the certain models can include a rigid mid-distal section which, although it can provide rigidity to protrude out of the skull, can also impede the flexibility of the device and cause the device to stick out beyond the scalp, making it more difficult to move the patient.

An intraventricular catheter can be inserted through a hole drilled into the skull, and passed through the dura mater and into the lateral ventricle of the patient's head, where CSF is held.

However, all of these methods are performed in a hospital setting, e.g. by a neurosurgeon, as they require many different surgical devices, a sterile environment to shave, clean and surgically implant the devices, and large external monitors within patient rooms. Many of approaches also are also very invasive and potentially traumatic because they involve a device contacting or passing through the dura matter.

A need exists for ICP measurement methods that can be used for head injury patients in non-hospital settings, including but not limited to office, field, battlefield, accident site, or ambulance settings. The use of pressure wire technology, similar to intravascular pressure-measuring guidewires (e.g., similar to the Philips Omniwire), would offer a small, minimally invasive device that could be used outside of a hospital (e.g., by paramedics) to measure ICP in trauma patients.

An intracranial guidewire can provide good pressure accurate readings, which allows for continual ICP monitoring within hospitals for patients with TBI. This can then assist clinicians in making treatment decisions. For example, patients with ICP values greater than 20 mmHg often require treatment, while ICP readings greater than 40 mmHg indicate a severe and potentially life-threatening condition.

Current market ICP devices have disadvantages that do not allow them to be easily utilized outside hospitals. The lumbar CSF pressure method inserts a catheter into the spinal cord-lumbar area, which often contradicts training directing emergency medical professionals to place victims on their back and to not manipulate the spinal area. Furthermore, CSF pressure in the lumbar region pressure does not directly correlate to ICP within the brain. Other devices such as the subdural bolt and intraventricular catheters are highly invasive devices and require additional highly advanced technological clinical devices to ensure that the bolt and catheters go into the brain tissue correctly to prevent additional brain damage. These types of devices, with their associated equipment, can also be very large and heavy which is not ideal for the use within emergency vehicles or to carry in medical bags.

To accurately measure ICP, a device can be placed beneath the skull, close to the ventricles, to measure the pressure buildup of the CSF. However, the intraventricular catheter penetrates directly through the brain structures, which, outside a hospital setting, may not be not safely possible without other equipment that is only found within hospitals and/or the backup of a neurology team standing by. These catheters are generally larger in diameter than a guidewire, which can reduce the maneuverability compared to a guidewire. The larger diameter increases the amount of brain tissue that experiences pressure as the devices is inserted into the brain to the ventricles. The more tissue the device interacts with and impacts, the larger the impact to neuro tissue and therefore possible impact to other motor and bodily functions. Catheters can also be more stiff/less flexible due to either the plastic material chosen and/or the overall diameter, which also reduce the maneuverability compared to a guidewire. Therefore, a device that is less invasive and smaller in profile is needed in order to reduce the risks of implanting the device.

In accordance with at least one embodiment of the present disclosure, an intracranial pressure sensing guidewire is provided which provides an alternate method for detecting and measuring intracranial pressure, through the use of a pressure monitoring transducer wire. This may for example be similar in structure to the Philips Omniwire (iFR) is a solid-core pressure-sensing guidewire that is intended to measure pressure in blood vessels, and to assist in the placement of catheters as necessary during procedures. However, an intravascular guidewire needs to have a proximate portion that is stiff enough to push through tortuous pathways of the vascular system, and a distal portion flexible enough to navigate through these pathways. The intravascular guidewire may also have its pressure sensor located at its distal tip. These features may not be suitable for an intracranial pressure sensing guidewire which needs to measure CSF pressure inside the skull with minimal risk of damage to brain tissue.

In that regard, an Omniwire-like pressure-sensing wire could be placed subdural, or near a location containing CSF, to measure ICP. Unlike the catheters used in hospitals to remove the built-up CSF, and unlike epidural sensors with significantly larger diameters, the intracranial wire would provide a smaller diameter, a more flexible material, and may not require placement into the brain tissue but instead merely within the skull proximate to the brain. In some aspects, the design of the wire could facilitate placement using an ultrasound machine or other imaging device. The intracranial pressure sensing guidewire would then allow for the ICP to be displayed on portable screens with technology that may, in some cases, already be available (e.g., similar to hardware currently used for measuring blood pressure).

Element(s) of the invention include:

A disposable, flexible, solid core pressure-sensing wire, used to measure pressure within the cranial cavity after a traumatic brain injury.

In an example, the pressure wire comes in only one available diameter, in order to reduce unnecessary space and weight occupied if multiple diameters are available. This also allows for simple sterile packaging, easy identification by the emergency medical personnel, and universal patient use. The wire may for example be formed of a lightweight, durable, biocompatible material that can remain in the body for an extent of time, such as Nitinol. The wire may have a rounded, padded, and/or spring-mounted tip, to limit the possibility of puncturing brain tissue. The working length of the wire may also have a relatively stiff proximal portion, allowing for maneuverability under the skull, and a more flexible distal portion that may, for example, allow the wire to be taped flat against the exterior of the head, without causing unwanted motion of the sensing portion located inside the skull. In an example, the pressure-sensing wire would be placed just under the skull, into the subarachnoid space and into the CSF that surrounds the brain and then connected proximally to a portable device to continually display the patient's ICP. In some aspects, if an increased ICP is detected, the wire could be used to assist in the placement of intraventricular catheters once the patient is admitted into a hospital, or more controlled environmental settings. The pressure wire would be able to guide the new catheter into the cranial cavity, similar to the way an intravascular guidewire can be used to assist in placement of an intravascular catheter for further treatment.

A display monitor. In an example, the display monitor is similar in size to a portable ultrasound monitor. The display monitor would be lightweight, small in dimensions, and made of durable materials.

A processor circuit in the display monitor that executes ICP monitoring software.

A self-stop cranial drill. Such a drill can be used to gain access under the skull, but may additionally have a component similar to an intraosseous needle (e.g., a needle for insertion through bone) that can be added over the drill bit to help with inserting and securing the pressure wire inside the skull.

The distal tip of the pressure-sensing wire may be similar to that of intravascular pressure-sensing guidewires, e.g., rounded, flexible, atraumatic tip to prevent tissue perforation. Proximal edges of the distal tip would need to be flush with the next device component and could be soldered or welded to the shaping section (e.g., a spring), which may be important as the device will be placed next to brain tissue. The material of the tip needs to be able to withstand a significant time period within CSF or near brain tissue. This is another difference from intravascular pressure-sensing guidewires, which may not be indicated for long-time use, as the pressure measurements can start to drift anywhere between 2 mmHg and 10 mmHg over a period of 30-120 minutes in the blood vessels, depending on the type of guidewire. The material of the tip, or portions thereof, may be selected to allow for visibility under portable ultrasound, which is a common device in trauma medical vehicles. This may for example allow the wire to be placed into CSF locations that are not under the skull, but accessible through other cranial areas such as the base of the skull.

A shaping section at or near the distal may be similar to the shaping coil used in intravascular guidewires. The coil may be flexible enough to support easy and safe placement under the skull, but durable enough to withstand significant time in the skull under pressures of 40 mmHg (0.8 pounds per square inch) or higher. For example, the coil section may need to withstand being pushed against skull bone by CSF pressure. The coil may also need to resist movement under the skull and in the CSF over time, and to be made of a biocompatible material that can be safely deployed in CSF and near brain tissue.

The working length and proximal core of the pressure-sensing wire must be stiff enough to allow for navigation into the correct ICP measuring location and for control when placed inside the cranial cavity, potentially increasing in durometer as it goes proximal. In an example, the wire may need to withstand bending with the radius of the cranium. The working length should have a diameter as small as possible in order to limit the access point size to insert into the patient and the amount of foreign material within the cranium. The working length can be relatively shorter (compared to an intravascular guidewire) to insert into the cranial area and connect to a portable pressure display monitor. In an example, the working length may be between 30 and 90 centimeters. A shorter wire requires less storage and use space, less device to have to maneuverer, and less weight added to the equipment of the emergency medical personnel.

The core of the pressure wire may be similar to that of an intravascular guidewire, to allow for the wire to maneuverer and be directed for specific placement. The material of the core is biocompatible, and selected to have a sufficient balance between stiffness and flexibility, to allow directing of the device while also allowing for ease of securing to the patient.

The pressure sensor may be equivalent or identical to the sensor used within a pressure-sensing guidewire such as the Philips Omniwire. Such a sensor measures blood pressure within vessels accurately (e.g., an average pressure of between 80 and 120 mmHg), and has been tested to a pressure limit of around 330 mmHg. This sensor can measure the ICP thresholds of 20 mmHg and 40 mmHg to quickly determine the right treatment path for a head trauma patient. Some pressure-sensing guidewires use two pressure sensors at the distal tip and then calculate the pressure difference based on the change between the two sensors over a vessel lesion. However, the ICP measurement wire needs to read the pressure inside the cranial cavity. Therefore, one sensor could be placed at the distal tip and then one towards the proximal end of the wire or in the display monitor to allow for the pressure difference to be measured, with a fairly simple design. The sensor may need to be calibrated and stabilized to prevent drifting of the reading over time if the intracranial guidewire is used to monitor the pressure for a relatively longer time (e.g., 24 hours or longer) such as in a search and rescue or combat situation where help is not immediately present. Critical monitoring time based on clinical studies is within the first 24 hours of a TBI. Increased pressure can occur after the first 24 hours, but most effects can be reversable within the first 24 hours. The sensor may also need to withstand the pressure of CSF pushing the wire against the bone of the skull.

The software for the intracranial pressure sensing guidewire may for example be similar to Philips Omniwire pressure-sensing display technology or SmartMap pressure reading technology, with pressure information gathered at the distal tip being translated for display on the monitor as a pressure value. The software may for example generate an alert level when the pressure is reaching critical ICP values such as 20 mmHg or 40 mmHg. The software can allow for specific programming of a critical ICP value, so that for example if a medical organization wanted to know when trauma victims reach a specific value between the averages of 20 mmHg and 40 mmHg, such an alert level could be set. The software can also be configured to minimize drifting of pressure readings over time.

The casing of the monitor is configured so that the device is reusable, with materials able to withstand sterile cleanings that are performed on monitoring systems such as portable ultrasound machines. Material of the casing may also be durable enough to protect the screen and electrical software components within the device. The casing may have a shape and texturing that allow for easy gripping of the device, and may include a clipping or hanging device to help secure the monitor to various fixtures in the area or ambulance.

The monitor screen is configured to display the pressure within the cranial cavity. The digital display may be large enough to be read easily in a moving ambulance or during trauma situations. The power supply can be configured to make the monitoring display device versatile to various trauma events (ambulance, search and rescue, combat, etc.). For example, there may be two power supply options: a plug port to connect the display to an external power supply (e.g., specific to ambulances or other emergency medical vehicles that have access to external power), or a battery pack located in the device (e.g., within the casing) that allows for a fully transportable system of display device and pressure-sensing wire. This may be appropriate for more remote traumas such as search and rescue or combat environments. The wire connection port in the display housing is configured to connect to a specific connector at the proximal end of the wire, to complete the circuit between the wire pressure sensor technology and the software of the monitor.

In some aspects, the intracranial pressure sensing guidewire may include a “Calibrate” button and “Start Reading” button. Instead of having the monitor read the pressure continuously once the wire is connected to the port, there may be a single button that, when pressed, starts the display of the pressure output. In some cases, the start button may double as a calibration button. Once the wire is connected but prior to insertion into the cranial cavity, the button can be pressed once until the reading is displayed at zero (balanced). Then the button can pressed again later to start reading pressure inside the skull. The intracranial pressure sensing guidewire may also include warning LEDs or indication lights that visually signal the state of ICP values and alert medical personnel when critical pressure levels are reached. In an example, two LEDs could be used: one green (below critical pressure levels), and one red (above critical levels), to provide redundancy and visual cues for pressure readings, in addition to a numerical display. The intracranial pressure sensing device may also include a sound alert system, similar in purpose to the LED indicators. To alert medical personnel to the rising ICP, the system may generate an alert sound should the pressure start reaching the critical alert pressure. Other user controls may be used instead or in addition, which can be actuated by the user (e.g., pressed, toggled, switched, etc.) to perform the same or similar functions to those described above.

The drill may include a drill bit with a diameter only slightly larger than the pressure wire diameter. Similar to intraosseous (IO) drills, there may be an access and securing channel (IO drills have this to allow for securing an IV line to the needle). The channel may for example have a tapered top to allow for easy access and insertion of the pressure wire through the drill bit and into the intracranial space. The taper would decrease until the diameter of the channel is only slightly larger than the drill bit and pressure wire diameters. The tighter tolerance of the channel to the wire can allow for securing of the wire to the channel, to support and hold the wire in place. The base of the channel may be designed to fit within the diameter of the burr hole created in the skull. It may be extremely important that the height of the base not extend beyond the skull bone and into brain tissue. At the top or bottom of the channel there may be a hemostasis valve, to ensure that CSF and blood do not flow out of the channel, and that external material does not enter the cranial cavity. The drill may for example be a self-stopping drill. Currently, trauma individuals and vehicles commonly carry an intraosseous drill in case access for IVs can only be obtained through the bone and into bone marrow. Access to the cranial cavity through burr holes can be obtained through a modified IO drill or through current self-stopping cranial drills.

The intracranial guidewire may be used outside of hospital settings where traumatic brain injuries are common, and therefore a greater concern may exist for increased ICP, and may thus need to be monitored quickly. Once a traumatic brain injury is identified through visual head wounds or is established as a possibility through analysis of the situation, access must be gained. A trained emergency medical individual (EMI) can establish access at two potential locations: near the area of injury, which would be more prone to increased pressure and greater brain trauma, or near an area with CSF to monitor any pressure build up. Once any critical injuries are addressed, any foreign objects are removed from the access point area, and the area is sterilized and cleaned, the EMI will open the sterile packaging using sterile practices.

The pressure wire may for example be packaged in a small sterile barrier pouch as an emergency tool that can be carried in a trauma medical kit. The majority of the intracranial guidewire's volume and weight may come from the display monitor, which may be designed to be as compact and portable as possible. The proximal end of the wire will be connected to the portable display monitor and the monitor turned on. The EMI may have to ensure that the initial reading is 0 mmHg, otherwise the ICP pressure read out may be incorrect. This could be addressed through the design of a calibration button to balance it in the field without the need of additional calibration equipment. During this step, the distal end of the wire may remain in the packaging. The wire may be packaged into a containment hoop to keep the distal tip of the intracranial guidewire sterile. Once connection to the monitor is confirmed and an accurate, balanced pressure readout is seen, access to the cavity may be performed.

Cranial cavity access will depend on the environment, patient condition, and EMI preference. Due to the small diameter of the intracranial guidewire compared to other monitors like the ICP bolt, the intracranial guidewire is easier to carry and will require less advanced technology and systems to accurately place the intracranial guidewire. If a breach through the skull is present due to injury, the EMI could gain access through the breach if the area is visible and clear. This can minimize the need to gain access elsewhere, although there may be higher risk of external material entering the wound, as well as CSF and blood exiting the cranial cavity. Should the EMI choose this access point, the area may need to be cleared of all visible barriers (bone, blood clots, etc.).

The length of the wire to be inserted can be determined outside of the skull by approximately noting the distance from the access point to where the EMI wants the wire tip. The pressure-sensing wire may then be fully removed from the packaging, and the distal tip placed through the breach in the bone, maneuvered as much as possible to the area of interest and secured to the patient's skin/head.

In other cases, a hole through the skull may be used. This can be done using an access drill and channel plug. The location of a burr hole may be extremely important. The closer to the injury site a burr hole is, the greater chance of measuring the pressure accurately. However, head injuries can be near vital areas of the brain where creating a burr hole may be inadvisable. Once a location is chosen to create a burr hole and is cleaned, the EMI can use the self-stopping drill and break through the skull to the CSF and brain cavity. Most EMIs already carry an IO drill, so the design of the wire and plug may be configured to be compatible with the drill that is already in medical trauma bags. After the drill has stopped (e.g., pressure against the drill bit is released), the channel plug will be placed into the hole and secured to the patient with medical tape, or gauze and pressure. Once the channel is secured, the wire can be removed fully from the packaging. As stated above, the length of the wire to be inserted will be determined by approximately noting the distance from the access point to where the EMI wants the wire tip. The distal tip is then inserted into the channel of the plug, through the hemostasis valve, and maneuvered to the location of interest chosen. The wire can then be secured to the channel.

In other cases, a needle can also be used to access locations of CSF near the base of the skull. This may eliminate the need to create an additional hole in the skull, but may not be close to the area of injury were pressure is more common to increase, and where pressure measurements will be more accurate. The EMI can then gain access using a gauged IV needle that allows for the wire to fit through it. The distal tip of the pressure wire may then be removed from the packaging and guided into the needle. Once the distal tip of the wire is within the CSF and pressures are being displayed on the monitor, the needle and wire can be secured to the patient with medical tape.

The pressure readings from the senor may then be displayed on the portable monitor, which can also be secured to the patient, to the trauma board, or to monitor display racks in a medical vehicle as the patient is treated and transported. Once the patient has reached a hospital or medical facility, the monitor can then be disconnected from the pressure wire. Based on the patient's medical status and the treatment chosen, the wire may then be used to assist in the placement of intraventricular catheters, or more advanced and larger monitoring devices, once the patient is admitted. The pressure wire can be used to guide the new devices into the cranial cavity and to an area with an increase in ICP.

The present disclosure aids substantially in the assessment of head trauma patients in non-hospital settings, by improving the ability of emergency medical personnel to obtain direct measurements of intracranial pressure. Implemented with a wire-mounted pressure sensor in communication with a portable monitor device, the intracranial pressure sensing guidewire disclosed herein provides practical improvements in the capabilities of emergency medical personnel. This improved methodology transforms a subjective, imprecise, symptom-based and demography-based assessment into a numerical reading indicative of the health status of the patient, and of interventions that may need to be taken. This occurs without the normally routine need to wait until the patient reaches the hospital and can be implanted with large, invasive intracranial sensors. This unconventional approach improves the functioning of an ambulance or other emergency vehicle, by giving its personnel access to more, and more accurate, information about the patient.

The intracranial pressure sensing guidewire may be implemented at least partially as a process viewable on a display, and operated by a control process executing on a processor that accepts user inputs from a keyboard, mouse, buttons, or touchscreen interface, and that is in communication with one or more sensors. In that regard, the control process performs certain specific operations in response to different inputs or selections made at different times. Outputs of the shown on a display, indicated with warning lights, audible tones, or haptic vibrations, or otherwise communicated to human operators. Certain structures, functions, and operations of the processor, display, sensors, and user input systems are known in the art, while others are recited herein to enable novel features or aspects of the present disclosure with particularity.

These descriptions are provided for exemplary purposes only, and should not be considered to limit the scope of the intracranial pressure sensing guidewire. Certain features may be added, removed, or modified without departing from the spirit of the claimed subject matter.

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

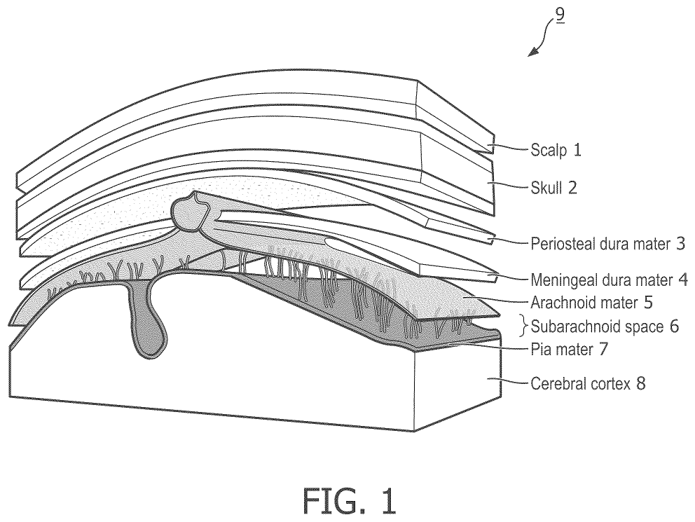

FIG. 1 is a schematic, diagrammatic representation of at least a portion of a human head or cranium 9, in accordance with aspects of the present disclosure. The cranium 9 is formed by the skull 2, which is covered by the scalp 1. Beneath the skull are the periosteal dura mater 3 and meningeal dura mater 4. Below the meningeal dura mater 4 (and interpenetrating with the meningeal dura mater 4 in some places) is the arachnoid mater 5. A fluid-filled space called the subarachnoid space 6 occurs between the arachnoid mater 5 and a layer called the pia mater 7, which covers the outer surface of the cerebral cortex 8. When a head trauma occurs, swelling of the brain (e.g., of the cerebral cortex 8 or of structures below or internal to the cerebral cortex 8) can increase the fluid pressure within the subarachnoid space 6 as described above.

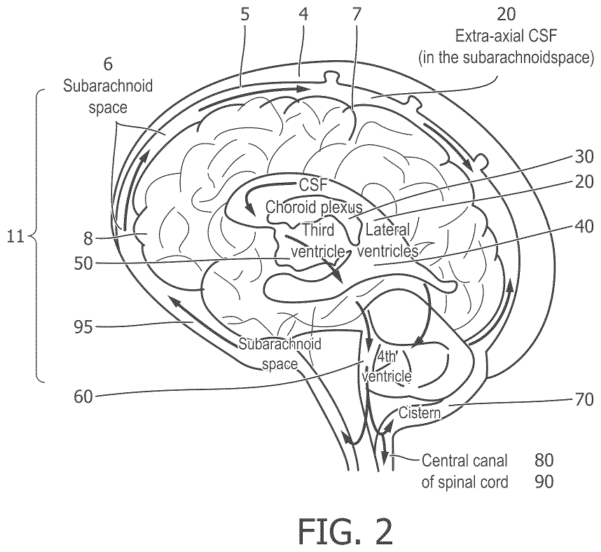

FIG. 2 is an exemplary representation of at least a portion of a human brain 11, in accordance with aspects of the present disclosure. Visible are the meningeal dura mater 4, arachnoid mater 5, pia mater 7, and subarachnoid space 6. The subarachnoid space is filled with extra-axial cerebrospinal fluid (CSF) 20. Other structures in the brain also contain CSF 20, including but not limited to the choroid plexus 30, lateral ventricles 40, third ventricle 50, fourth ventricle 60, cistern 70, and the central canal 80 of the spinal cord 90. White arrows 95 indicate the direction of flow of the CSF 20. Damage to the brain 11 can cause swelling, leading to an increase in the pressure of the CSF 20. For example, swelling of the brain 11 can compress the cerebral cortex 8 and pia mater 7 into the arachnoid mater, thus decreasing the volume of the subarachnoid space 6, which then causes the pressure of the CSF 20 within the subarachnoid space 6 to increase.

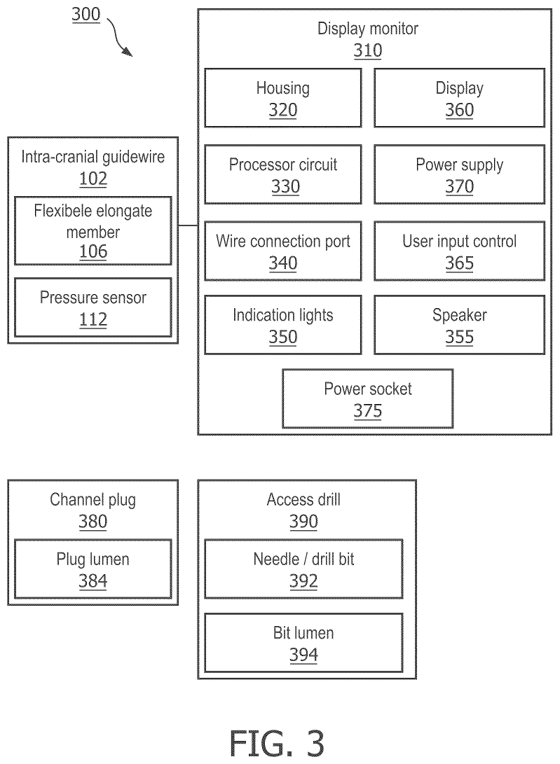

FIG. 3 is a schematic, diagrammatic view, in block diagram form, of an intracranial pressure sensing system 300, according to aspects of the present disclosure. In the example shown in FIG. 3, the intracranial pressure sensing system 300 includes an intracranial sensing device or intracranial guidewire 102, a display monitor 310, a channel plug 380, and an access drill or intraosseous drill 390. The intracranial guidewire 102 includes a flexible elongate member 106 (e.g., a guidewire) and a pressure sensor 112. The intracranial guidewire 102 connects to, receives power from, and sends sensor readings to, the display monitor 310, such that when the pressure sensor 112 is inside the skull (e.g., positioned within the subarachnoid space), the pressure sensor 112 detects the pressure of the cerebrospinal fluid (CSF).

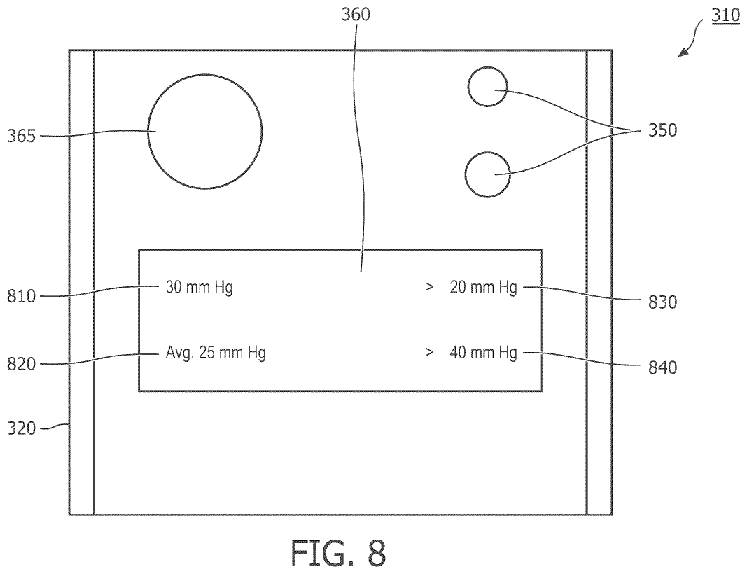

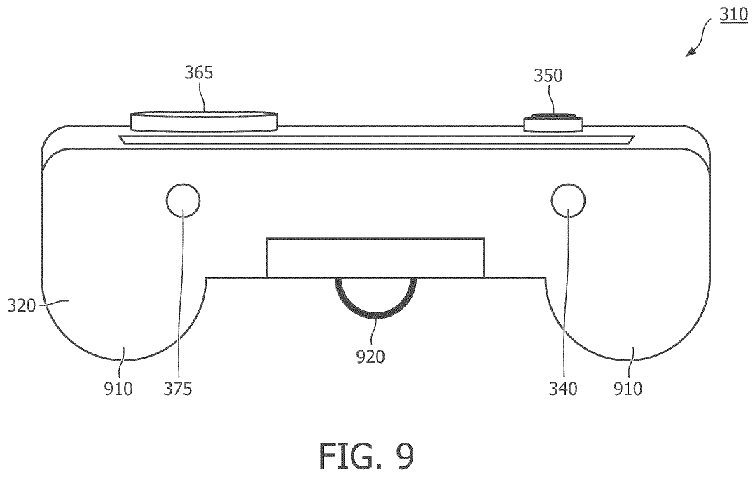

The display monitor 310 includes a housing, case, or casing 320, a processor circuit 330, a guidewire connection port or receiver portion 340, indication lights or indicator lights 350, a display 360, a power supply 370 (e.g., a replaceable or rechargeable battery), a user input control 365 (e.g., a button, switch, dial, touchscreen, etc.), a speaker 355, and a power socket 375 (e.g., for receiving a power cord or charging cable). In an example, the display monitor 310 received pressure readings from the pressure sensor 112 via the guidewire connection port 340, processes the pressure readings with the processor circuit 330 to determine the pressure of the CSF, and then displays the results on the display 360 and/or indication lights 350. Operation of the display monitor 310 is described below in further detail, e.g. in FIGS. 8 and 9.

The channel plug 380 can be inserted into a burr hole in the skull, such that a portion of the channel plug 380 is outside of the scalp and another portion of the channel plug 380 is inside the skull (e.g., inside the subarachnoid space 6). The channel plug 380 includes a channel plug lumen 384 through which the intracranial guidewire 102 can be extended, such that a portion of the intracranial guidewire 102 (e.g., a portion including the pressure sensor 112) is advanced into a space that contains CSF.

The access drill 390 includes a hollow needle or drill bit 392 that can be used to form a burr hole in the skull. The needle or drill bit 392 includes a bit lumen 394 through which the intracranial guidewire 102 can be threaded, when detached from the drill head, to facilitate insertion of the intracranial guidewire 102 through the channel plug 380 and into the interior of the skull.

It is noted that block diagrams are provided herein for exemplary purposes; a person of ordinary skill in the art will recognize myriad variations that nonetheless fall within the scope of the present disclosure. For example, block diagrams may show a particular arrangement of components, modules, services, steps, processes, or layers, resulting in a particular flow of power, information, etc. It is understood that some aspects of the systems disclosed herein may include additional components, that some components shown may be absent from some embodiments, and that the arrangement of components may be different than shown, resulting in different data flows while still performing the methods described herein.

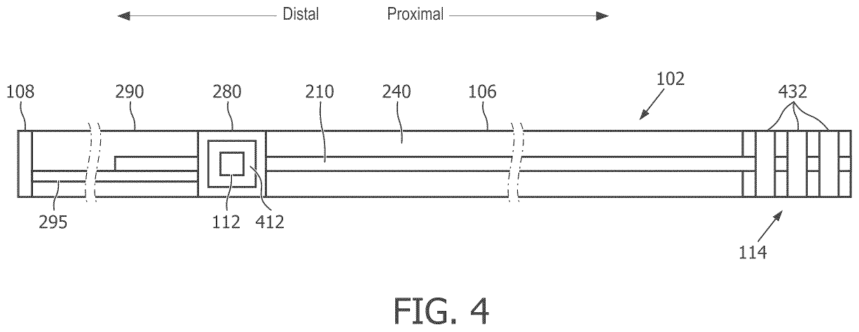

FIG. 4 is a diagrammatic cross-sectional view of an intracranial sensing guidewire 102 that includes a pressure sensor 112, according to aspects of the present disclosure. The intracranial sensing guidewire 102 is configured for insertion into the CSF (e.g., into the cranium via a burr hole in the skull). In the example shown in FIG. 4, a pressure sensor 112 positioned proximal of a distal end of the flexible elongate member 106. The pressure sensor 112 may for example be planar, circumferential, or otherwise, and may be a piezoelectric sensor, a capacitive micromachined ultrasound transducer (CMUT) sensor, or otherwise. The pressure sensor 112 may be at least partially contained within a sensor housing 280 and/or a sensor mount 412. A coil 290 is disposed distal of the pressure sensor housing 280, and provides additional flexibility to the distal portion of the pressure-sensing intracranial guidewire 102. A shaping ribbon or other shaping member 295 may be located within the coil and fixedly attached to a core member or core wire 210. The shaping ribbon 295 may for example allow a clinician to bend the coil 290 into a hook shape, or other desired shape, e.g., to facilitate navigation of the guidewire within the interior of the skull. The core wire 210 forms the central structure or structural support of the flexible elongate member 106, and may for example be made of a metal such as stainless steel or nitinol, and may extend from the coil 290 to the proximal end of the flexible elongate member 106. In some instances, at least some portions of the core wire 210 may be surrounded by a polymer layer 240, although this may not be the case for the portion of the core wire 210 located within the coil 290.

At the distal end of the flexible elongate member 106 is a tip 108, which may for example be a rounded or atraumatic tip made of metal or polymer. At the proximal end of the flexible elongate member 106 is a connection portion 114 that includes a plurality of conductive portions 432, such as circumferential conductive bands. In an example, the pressure sensor 112 may be operated by three conductor paths, such as three conductive wires or filars, or two conductive wires or filars plus a conductive core wire. Thus, three of the conductive bands 432 may be electrically connected to the pressure sensor 112. It is noted that the conductive wires or filars could have circular cross section or a flatted ribbon-like cross section, or other cross section, and could comprise multiple segments of different cross section and/or different materials.

In some aspects, one of the wires or filars would be grounded (e.g., to the core wire) and thus not need a conductive band. Thus, there may be two or three conductive bands 432 in the connection portion 114. Depending on the implementation, other numbers of conductive bands or conductive paths, and/or other wiring or connection methods, may be used instead or in addition.

Other numbers or arrangements of sensors may also be used. In an intracranial sensing guidewire that includes a first sensor and a second sensor, the first sensor can include any suitable sensing modality (e.g., pressure, temperature, imaging, etc.). The second sensor can also be any suitable modality (e.g., pressure, temperature, imaging, etc.), whether the same or different than the modality of the first sensor. In the example shown in FIG. 4, the flexible elongate member of the intracranial sensing guidewire does not include a lumen.

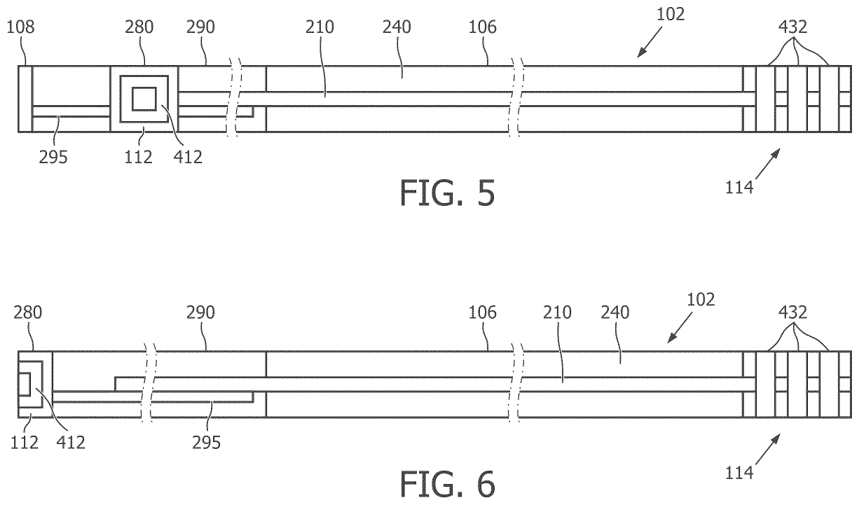

FIG. 5 is a diagrammatic cross-sectional view of an intracranial sensing guidewire 102 that includes a pressure sensor 112, according to aspects of the present disclosure. Visible are the tip 108 and coil 290 of the flexible elongate member 106, along with the shaping ribbon 295, core wire 210, polymer layer 240, connection portion 114, and conductive bands 432. This configuration is similar to that of FIG. 4, except that the pressure sensor 112, including the pressure sensor housing 280 and/or pressure sensor mount 412, is located within the coil 290. This may, for example, provide greater protection to the pressure sensor 112 than in the configuration shown in FIG. 4. Also, like FIG. 4, this configuration advantageously avoids measuring the pressure of the tip 108, when the tip 108 is pressed against a surface, rather than measuring the pressure of the surrounding CSF. In some aspects, an aperture may be formed in the coil 290 to facilitate pressure measurement by the sensor 112, although this may not be necessary in all cases.

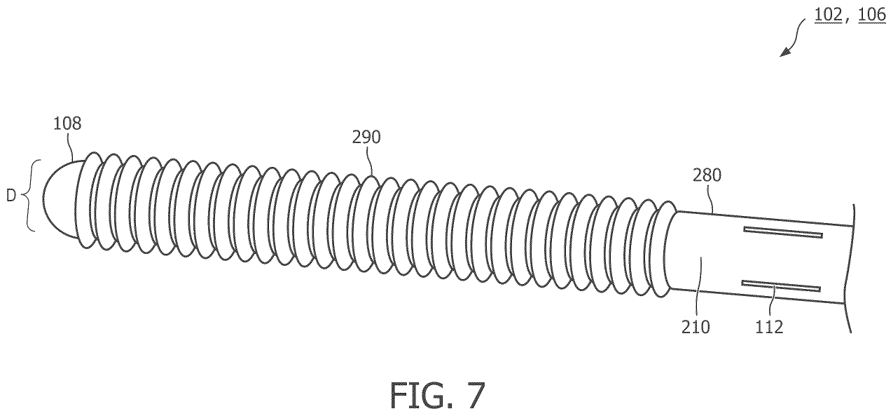

FIG. 6 is a diagrammatic cross-sectional view of an intracranial sensing guidewire 102 that includes a pressure sensor 112, according to aspects of the present disclosure. Visible are the coil 290 of the flexible elongate member 106, along with the shaping ribbon 295, core wire 210, polymer layer 240, connection portion 114, and conductive bands 432. This configuration is similar to that of FIG. 4, except that the pressure sensor 112, including the pressure sensor housing 280 and/or pressure sensor mount 412, is located at the distal end of the flexible elongate member 106, in place of the tip 108. This configuration advantageously allows the pressure sensor 112 to measure the pressure of the CSF while extended a shorter distance into the CSF (e.g., into the sub-arachnoid gap) than would be possible with the configurations of FIGS. 4 and 5.

FIG. 7 is a side perspective cross-sectional view of an intracranial pressure sensing device 102 (e.g., a pressure-sensing guidewire) that includes a pressure sensor 112, according to aspects of the present disclosure. Visible are the tip 108, coil 290, and core wire 210 of the flexible elongate member 106, along with the sensor housing 280 and pressure sensor 112. In the example shown in FIG. 7, both the sensor housing 280 and the pressure sensor 112 are circumferential (e.g., encircling the core wire). It is understood that other configurations (e.g., planar, multiple sensors, etc.) may be used instead or in addition. The intracranial pressure sensing guidewire 102 has a diameter D that may for example fall within the range of 0.014-0.035 inches (0.36 to 0.89 cm). Thus, the guidewire 102 can potentially be inserted into the skull through a burr hole of comparable diameter to the intracranial guidewire 102 itself. This represents a substantial improvement over the current art, as insertion of a subdural pressure-sensing catheter into the skull typically requires a burr hole diameter of 3.5 to 11.5 cm, depending on the type of catheter, while insertion of a subdural pressure-sending bolt can require a burr hole diameter of up to 1 cm in diameter. Such large holes are highly invasive, expose more brain tissue to the external environment, and may not be capable of natural healing, and may thus need to be covered with metal plate once treatment of the head injury is complete. Conversely, the smaller burr hole of the present disclosure may be less invasive to the patient, and may be capable of self-healing to full closure.

FIG. 8 is a diagrammatic cross-sectional view of a display monitor 310, according to aspects of the present disclosure. Visible are the housing 320, indicator lights 350, user control 365 (e.g., a button), and display 360. In the example shown in FIG. 8, the display 360 shows an instantaneous intracranial pressure reading 810, which may for example be a reading of the pressure of the CSF in the sub-arachnoid space. However, instantaneous pressure readings may vary due to factors such as heartbeat, body position or motion, vibration of a vehicle transporting the patient, etc. Thus, in some aspects the display 360 may, instead or in addition, show a time-averaged intracranial pressure reading 820 (e.g., an average of instantaneous pressure readings over a period of 1 second, 5 seconds, 10 seconds, etc.).

In an example, a user may plug the intracranial pressure-sensing guidewire into the display monitor 310, activate the user control 365 (e.g., press a button) in order to zero or calibrate the sensor to atmospheric pressure, at which point a first indicator light 350 (e.g., a green LED) will illuminate, to indicate that the guidewire is plugged in and the display monitor 310 is receiving pressure measurements from it. The guidewire can then be inserted into the skull through a burr hole.

Certain pressure thresholds are considered to be of particular medical significance. For example, an intracranial pressure of 20 mmHg may be indicative that the patient requires treatment to reduce the pressure of the intracranial CSF, while a pressure of 40 mmHg may indicate a severe condition requiring more immediate or more drastic intervention. Thus, the display 360 may also include a first threshold warning 830 (e.g., a display of alphanumeric characters warning that the intracranial pressure is greater than 20 mmHg—highlighted in the example of FIG. 8, since the measured pressure is above this threshold) and a second threshold warning 840 (e.g., a warning that the intracranial pressure is greater than 40 mmHg—grayed in the example of FIG. 8, since the measured pressure is less than this threshold). Depending on the implementation, other threshold indications may be provided instead or in addition. For example, a second indicator light 350 (e.g., a red LED) may illuminate if the sensed pressure exceeds a first threshold value (e.g., 20 mmHg), and may blink if the sensed pressure exceeds a second threshold value (e.g., 40 mmHg). In some aspects, an audible alarm may be used. For example, an audio speaker may emit a soft tone or series of beeps if the pressure is below the first threshold value, may emit a louder, lower tone or a faster series of beeps if the pressure is above the first threshold value, and may emit a still louder, lower tone or a still faster series of beeps if the pressure is above the second threshold value.

Depending on the implementation, feedback to the user could also include haptic feedback (e.g., vibration), graphs or moving images on the display 360, or other means as would occur to a person of ordinary skill in the art. In some aspects, the display monitor 310 may include an option for the user to change the threshold values. The pressure values described above may be shown in any suitable units, including millimeters of mercury (mmHg), pounds per square inch (psi), megapascals (mPa), millibars, etc. In some aspects, the display monitor 310 may include an option for the user to change the units.

FIG. 9 is a diagrammatic cross-sectional view of a display monitor 310, according to aspects of the present disclosure. Visible are the housing or case 310, indicator lights 350, and user control 365. In the example shown in FIG. 9, the housing 320 includes two ergonomic handgrips 910, which may facilitate holding or handling by the user, as well as a hook or hanger 920, which may facilitate hanging the display monitor 310 from a bed rail, IV pole, cart, hanger, etc. Other ergonomic features may be provided instead or in addition, without departing from the spirit of the present disclosure. The exemplary display monitor 310 of FIG. 9 also includes a guidewire connector port 340, into which the intracranial pressure-sensing guidewire can be inserted, and a power socket 375, into which a power cord or charging cable can be plugged (e.g., if the internal battery is depleted). In an example, when the connection portion 114 of the intracranial pressure-sensing guidewire 102 (see FIG. 4) is inserted into the guidewire connector port 340, the pressure sensor 112 (see FIG. 4) receives power from the display monitor 310 and sends pressure readings to the display monitor 310.

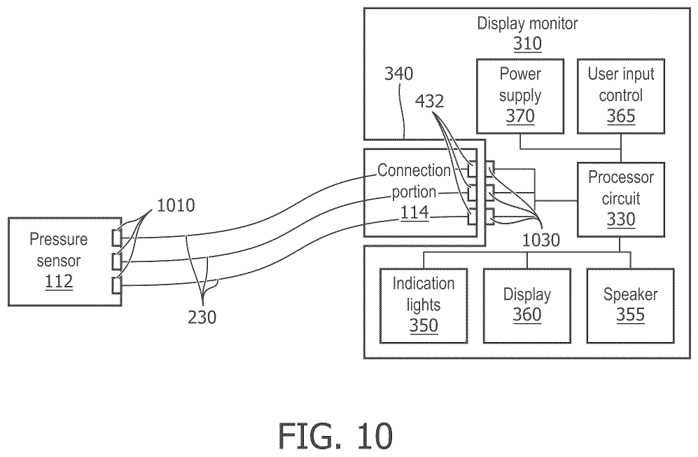

FIG. 10 is a schematic, diagrammatic view, in block diagram form, of the wiring of an intracranial pressure sensing device 102 that includes a pressure sensor 112, according to aspects of the present disclosure. The connection portion 114 of the intracranial device 102 includes a number of conductive portions 432 (e.g., conductive bands), to which wires, filars, or conductors 230 are electrically connected. The wires or filars 230 extend from the connection portion 114 to electrical contacts 1010 on the pressure sensor 112, in order to carry power and signals between the connection portion 114 and the pressure sensor 112. In an example, the wires or filars 230 are electrically bonded (e.g., by solder, conductive adhesive, ultrasonic welding, or other means) to the electrical contacts 1010.

One or a plurality of the wires, filars, or conductors 230 could be a continuous length or multiple wires/conductors that are electrically and mechanical coupled. For example, part of the length could be a filar e.g., extending along/wrapped around the core wire and part of the length could be, for example, a ribbon conductor extending along/embedded in polymer around the core wire. A wire or filar, or a portion thereof, could be a bare metal conductor that is surrounded by polymer insulation. Exposed portions of the bare conductors at the ends (e.g., portions without insulation) can make electrical contact.

Other numbers or routings of wires, filars, embedded conductors, or other conductive pathways may be provided instead of or in addition to those shown in FIG. 10, without departing from the spirit of the present disclosure. When the intracranial guidewire is plugged into the display monitor 310, the conductive portions 432 of the connection portion 114 are in electrical contact with conductive portions 1030 of the guidewire connection port 340, which receive electrical power from the power supply 370 and transfer signals to and from the processor circuit 330.

Also visible are the user input control 365, indication lights or indicator lights 350, display 360, and speaker 355, each of which is in electrical communication with the processor circuit 330.

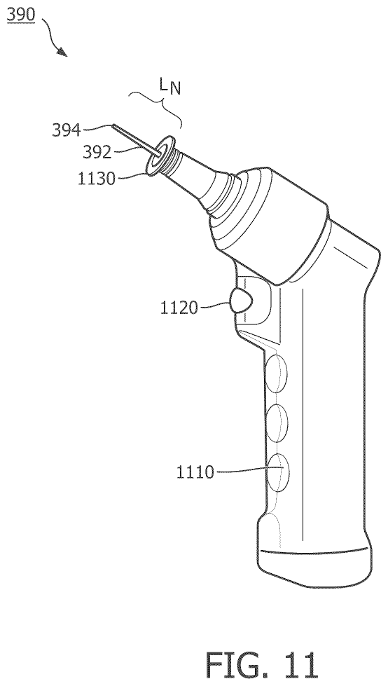

FIG. 11 is a side perspective view of an access drill or intraosseous drill 390, according to aspects of the present disclosure. The drill 390 is sized and shaped for operation in field settings by emergency medical personnel, and includes a handle 1110 and trigger 1120. When the trigger 1120 is pulled, the needle or drill bit 392 rotates at a speed sufficient to drill through bone and thus form a burr hole (e.g., through the skull). The drill 390 also includes a stop 1130 to prevent the needle or drill bit 392 from drilling past its exposed length Lx. In an example, Lx may be between 5 mm and 10 mm, to facilitate drilling completely through the scalp 1, skull 2, periosteal dura mater 3, meningeal dura mater 4, and arachnoid mater 5, and thus into the subarachnoid space 6, with minimal risk of puncturing the pia mater 7 or cerebral cortex 8 (see FIG. 1). In some aspects, the needle or bit 392 is hollow, and includes a bit lumen 394, which may facilitate the insertion of the intracranial pressure-sensing guidewire 102 into the skull through the burr hole. In an example, the intracranial guidewire 102 may be threaded through the bit lumen 394 while the bit 392 is still in the burr hole, and then the bit 392 may be removed from the burr hole, leaving the intracranial guidewire in place.

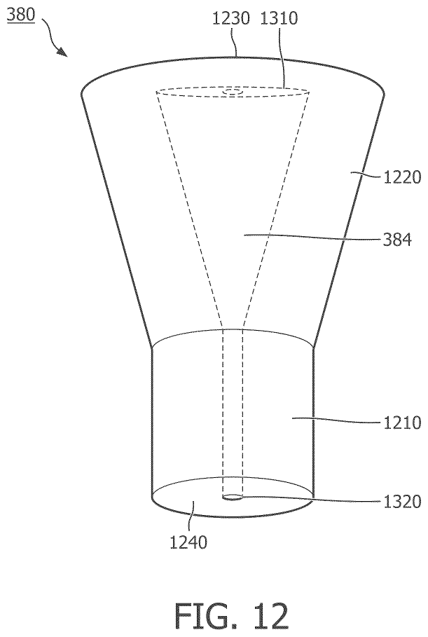

FIG. 12 is a side perspective view of a channel plug 380, according to aspects of the present disclosure. The channel plug 380 is configured to be inserted into a burr hole drilled in the skull. In the example shown in FIG. 12, the channel plug 380 includes a roughly cylindrical portion 1210, at least a portion of which may project through the underside of the skull when the channel plug 380 is fully inserted into the burr hole. The channel plug 380 also includes a tapered portion 1220, at least a portion of which may protrude above the scalp when the channel plug 380 is fully inserted into the burr hole. The channel plug also includes a channel plug lumen 384 extending completely through the channel plug 380 from the top surface 1230 to the bottom surface 1240. For example, the lumen 384 extends between a top opening 1310 at the top surface 1230 and a bottom opening 1320 at the bottom surface 1240. The channel plug lumen 384 may also include a taper, such that it is wider at the top surface 1230 than at the bottom surface 1240, to facilitate insertion of the intracranial guidewire 102 through the channel plug 380 via the channel plug lumen 384, and thus into the interior of the cranium (e.g., into the subarachnoid space). In the illustrated aspect, the lumen 384 can include with two sections: a first section is proximate/nearer to the top surface 1230 and a second section is proximate/nearer to the bottom surface 1240. The first section has a larger slope for the taper of the lumen 384, while the second section has a smaller slope for the taper of the lumen 384, which can provide the lumen 384 with a funnel shape (wider at the top surface 1230, narrower at the bottom surface 1240). The wider opening for the lumen 384 advantageously provides easier insertion of the guidewire at the top surface 1230, while the narrower opening at the bottom surface 1240 advantageously guides the guidewire inside the skull (e.g., into the subarachnoid space). It is understood that the shape of the channel plug 380 and channel plug lumen 384 may be different than shown herein, while performing the same or a similar function.

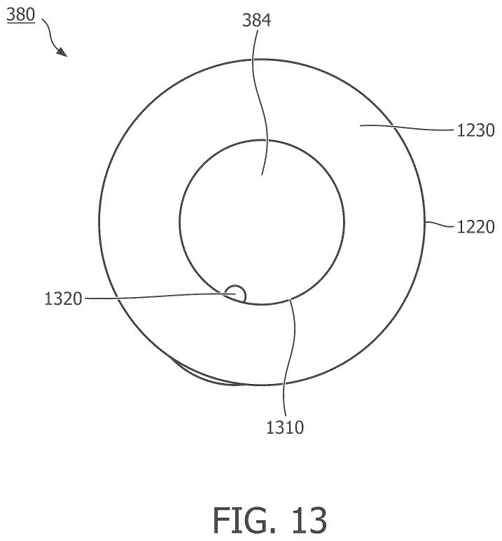

FIG. 13 is a top perspective view of a channel plug 380, according to aspects of the present disclosure. Visible are the top surface 1230, tapered portion 1220, and channel plug lumen 384. In the example shown in FIG. 13, the taper of the channel plug lumen 384 is more pronounced than in the example of FIG. 12, with the top opening 1310 being significantly larger than the bottom opening 1320. Depending on the implementation, various diameters of the top opening 1310 and bottom opening 1320 may be used, including positive, negative, and zero tapers, without departing from the spirit of the present disclosure.

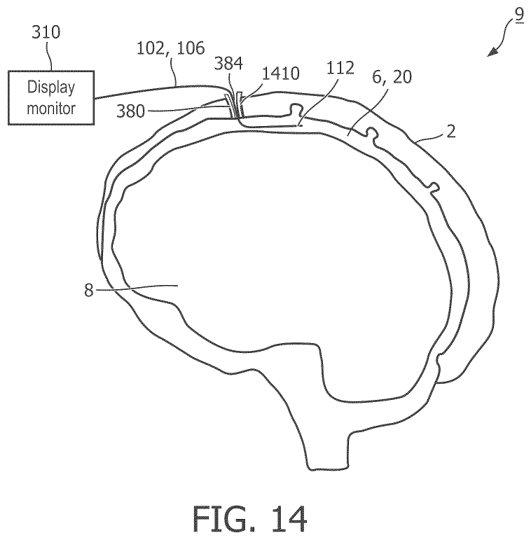

FIG. 14 is a diagrammatic, side cross-sectional view of an intracranial pressure sensing device 102 (e.g., an intracranial pressure-sensing guidewire) inserted into the subarachnoid space 6 of a human cranium 9, according to aspects of the present disclosure. In the example shown in FIG. 14, a burr hole 1410 has been drilled completely through the skull 2 and into the subarachnoid space 6. A channel plug 380 has been inserted through the burr hole, such that the channel plug lumen 384 extends from the exterior of the cranium 9 into the subarachnoid space 6. Next, the flexible elongate member 106 of the intracranial pressure sensing guidewire 102 is inserted through the channel plug lumen 384 until the pressure sensor 112 is fully within the subarachnoid space 6. In this configuration, pressure measurements taken by the pressure sensor 112 may be indicative of the pressure of the CSF 20 in the subarachnoid space. Since the intracranial pressure sensing guidewire 102 is connected to the display monitor 310, the pressure of the CSF 20 is then displayed on the display monitor 310 for review by medical personnel.

It is noted that the most desirable orientation of the guidewire 102 and sensor 112 may be as shown, which may require navigating the tip of the guidewire such that it forms approximately a 90 degree bend below the channel plug lumen 384, to avoid contacting or damaging the tissues of the cerebral cortex 8. In some instances, this navigation may, for example, be facilitated by bending the shaping ribbon 295 (see FIG. 4) into a curved, hooked, or angled shape. However, in other cases, the flexibility of the guidewire may be such that it naturally bends away from the cerebral cortex 8 rather than pressing into it. For convenience and safety, it may be desirable for medical personnel (e.g., paramedics) to fix the exposed portion of the guidewire to the cranium, e.g., by gluing, taping, or stapling it to the scalp.

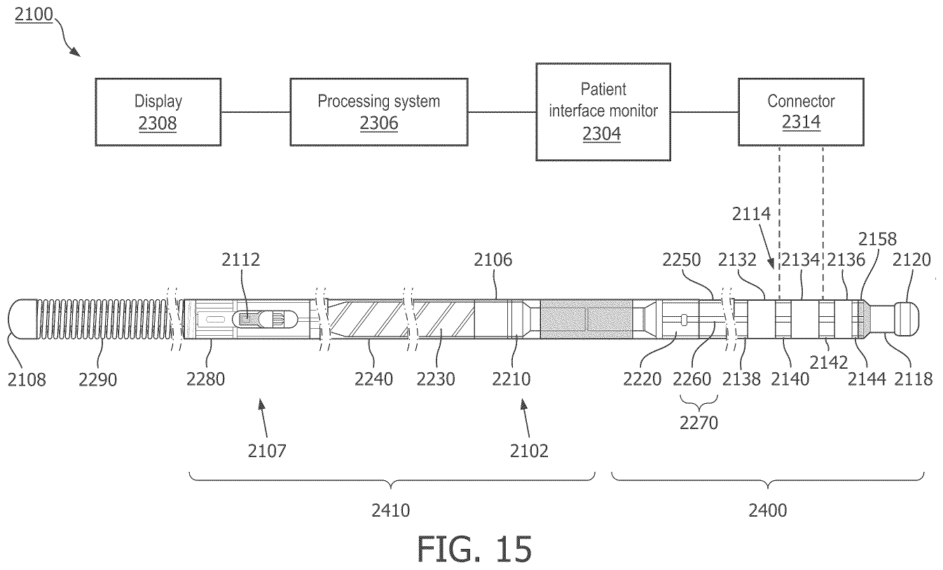

FIG. 15 is a diagrammatic side view of an intraluminal (e.g., intravascular) sensing system 2100 that includes an intravascular pressure-sensing device 2102, according to aspects of the present disclosure. The intraluminal sensing system 2100 may be similar in some ways to the intracranial sensing system 300 of FIG. 3, and the intravascular device 2102 may be similar in some ways to the intracranial pressure-sensing device 102 of FIG. 3.

The intravascular device 2102 can be an intravascular guidewire sized and shaped for positioning within a vessel of a patient. The intravascular device 2102 includes a distal tip 2108 and an electronic component 2112. For example, the electronic component 2112 can be a pressure sensor configured to measure a pressure of blood within the vessel of the patient, or another type of sensor including but not limited to a flow sensor, temperature sensor, or imaging sensor, or combination sensor measuring more than one property. The intravascular device 2102 includes a flexible elongate member 2106. The electronic component 2112 is disposed at a distal portion 2107 of the flexible elongate member 2106. The electronic component 2112 can be mounted at the distal portion 2107 within a housing 2280 in some embodiments. A flexible tip coil 2290 extends distally from the housing 2280 at the distal portion 2107 of the flexible elongate member 2106. A connection portion 2114 located at a proximal end of the flexible elongate member 2106 includes conductive portions 2132, 2134. A locking area is formed by a collar or locking section 2118 and knob or retention section 2120 are disposed at the proximal portion 2109 of the flexible elongate member 2106.

The intravascular device 2102 in FIG. 15 includes core wire comprising a distal core 2210 and a proximal core 2220. The distal core 2210 and the proximal core 2220 are metallic components forming part of the body of the intravascular device 2102. For example, the distal core 2210 and the proximal core 2220 may be flexible metallic rods that provide structure for the flexible elongate member 2106. The distal core 2210 and/or the proximal core 2220 can be made of a metal or metal alloy. For example, the distal core 2210 and/or the proximal core 2220 can be made of stainless steel, Nitinol, nickel-cobalt-chromium-molybdenum alloy (e.g., MP35N), and/or other suitable materials. In some embodiments, the distal core 2210 and the proximal core 2220 are made of the same material. In other embodiments, the distal core 2210 and the proximal core 2220 are made of different materials. The diameter of the distal core 2210 and the proximal core 2220 can vary along their respective lengths. A joint between the distal core 2210 and proximal core 2220 is surrounded and contained by a hypotube 2215. The electronic component 2112 can in some cases be positioned at a distal end of the distal core 2210.

In some embodiments, the intravascular device 2102 comprises a distal subassembly 2410 and a proximal subassembly 2400 that are electrically and mechanically joined together, which creates an electrical communication between the electronic component 2112 and the conductive portions 2132, 2134. For example, flow data obtained by the electronic component 2112 (in this example, electronic component 2112 is a flow sensor) can be transmitted to the conductive portions 2132, 2134. In an exemplary embodiment, the pressure sensor 2112 is a single pressure transducer element. In some embodiments, the transducer generates electrical signals representative of the pressure of the fluid within the vessel. The processing system 2306 processes the electrical signals to extract the pressure of the fluid. The signal-carrying filars carry these electrical signals from the sensor at the distal portion to the connector at the proximal portion.

Control signals from a processing system 2306 (e.g., a processor circuit of the processing system 2306) in communication with the intravascular device 2102 can be transmitted to the electronic component 2112 via a connector 2314 that attaches to the conductive portions 2132, 2134. The distal subassembly 2410 can include the distal core 2210. The distal subassembly 2410 can also include the electronic component 2112, the conductive members 2230, and/or one or more layers of insulative polymer/plastic 2240 surrounding the conductive members 2230 and the core 2210. For example, the polymer/plastic layer(s) can insulate and protect the conductive members of the multi-filar cable or conductor bundle 2230. The proximal subassembly 2400 can include the proximal core 2220. The proximal subassembly 2400 can also include one or more polymer layers 2250 (hereinafter polymer layer 2250) surrounding the proximal core 2220 and/or conductive ribbons 2260 embedded within the one or more insulative and/or protective polymer layer 2250. In some embodiments, the proximal subassembly 2400 and the distal subassembly 2410 are separately manufactured. During the assembly process for the intravascular device 2102, the proximal subassembly 2400 and the distal subassembly 2410 can be electrically and mechanically joined together. As used herein, flexible elongate member can refer to one or more components along the entire length of the intravascular device 2102, one or more components of the proximal subassembly 2400 (e.g., including the proximal core 2220, etc.), and/or one or more components the distal subassembly 2410 (e.g., including the distal core 2210, etc.). Accordingly, flexible elongate member may refer to the combined proximal and distal subassemblies described above. The joint between the proximal core 2220 and distal core 2210 is surrounded by the hypotube 2215.

In various embodiments, the intravascular device 2102 can include one, two, three, or more core wires extending along its length. For example, a single core wire can extend substantially along the entire length of the flexible elongate member 2106. In such embodiments, a locking section 2118 and a section 2120 can be integrally formed at the proximal portion of the single core wire. The electronic component 2112 can be secured at the distal portion of the single core wire. In other embodiments, such as the embodiment illustrated in FIG. 1, the locking section 2118 and the section 2120 can be integrally formed at the proximal portion of the proximal core 2220. The electronic component 2112 can be secured at the distal portion of the distal core 2210. The intravascular device 2102 includes one or more conductive members 2230 (e.g., a multi-filar conductor bundle or cable) in communication with the electronic component 2112. For example, the conductive members 2230 can be one or more electrical wires that are directly in communication with the electronic component 2112. In some instances, the conductive members 2230 are electrically and mechanically coupled to the electronic component 2112 by, e.g., soldering. In some instances, the conductor bundle 2230 comprises two or three electrical wires (e.g., a bifilar cable or a trifilar cable). An individual electrical wire can include a bare metallic conductor surrounded by one or more insulating layers. The conductive members 2230 can extend along the length of the distal core 2210. For example, at least a portion of the conductive members 2230 can be spirally wrapped around the distal core 2210, minimizing or eliminating whipping of the distal core within tortuous anatomy.

The intravascular device 2102 may include one or more conductive ribbons 2260 at the proximal portion of the flexible elongate member 2106. The conductive ribbons 2260 are embedded within polymer layer 2250. The conductive ribbons 2260 are directly in communication with the conductive portions 2132 and/or 2134. In some instances, a multi-filar conductor bundle 2230 is electrically and mechanically coupled to the electronic component 2112 by, e.g., soldering.

As described herein, electrical communication between the conductive members 2230 and the conductive ribbons 2260 can be established at the connection portion 2114 of the flexible elongate member 2106. By establishing electrical communication between the conductor bundle 2230 and the conductive ribbons 2260, the conductive portions 2132, 2134 can be in electrical communication with the electronic component 2112.

In some aspects represented by FIG. 15, the intravascular device 2102 includes a locking section 2118 and a retention section 2120. To form locking section 2118, a machining process is used to remove polymer layer 2250 and conductive ribbons 2260 in locking section 2118 and to shape proximal core 2220 in locking section 2118 to the desired shape. As shown in FIG. 15, locking section 2118 includes a reduced diameter while retention section 2120 has a diameter substantially similar to that of proximal core 2220 in the connection portion 2114. In some instances, because the machining process removes conductive ribbons in locking section 2118, proximal ends of the conductive ribbons 2260 would be exposed to moisture and/or liquids, such as blood, saline solutions, disinfectants, and/or enzyme cleaner solutions, an insulation layer 2158 is formed over the proximal end portion of the connection portion 2114 to insulate the exposed conductive ribbons 2260.

In some embodiments, a connector 2314 provides electrical connectivity between the conductive portions 2132, 2134 and a patient interface monitor 2304. The Patient Interface Monitor 2304 may in some cases connect to a console or processing system 2306, which includes or is in communication with a display 2308.

The system 2100 may be deployed in a catheterization laboratory having a control room. The processing system 2306 may be located in the control room. Optionally, the processing system 2306 may be located elsewhere, such as in the catheterization laboratory itself. The catheterization laboratory may include a sterile field while its associated control room may or may not be sterile depending on the procedure to be performed and/or on the health care facility. In some embodiments, device 2102 may be controlled from a remote location such as the control room, such that an operator is not required to be in close proximity to the patient.

The intraluminal device 2102, PIM 2304, and display 2308 may be communicatively coupled directly or indirectly to the processing system 2306. These elements may be communicatively coupled to the medical processing system 2306 via a wired connection such as a standard copper multi-filar conductor bundle 2230. The processing system 2306 may be communicatively coupled to one or more data networks, e.g., a TCP/IP-based local area network (LAN). In other embodiments, different protocols may be utilized such as Synchronous Optical Networking (SONET). In some cases, the processing system 2306 may be communicatively coupled to a wide area network (WAN).

The PIM 2304 transfers the received signals to the processing system 2306 where the information is processed and displayed (e.g., as physiology data in graphical, symbolic, or alphanumeric form) on the display 2308. The console or processing system 2306 can include a processor and a memory. The processing system 2306 may be operable to facilitate the features of the intravascular sensing system 2100 described herein. For example, the processor can execute computer readable instructions stored on the non-transitory tangible computer readable medium.

The PIM 2304 facilitates communication of signals between the processing system 306 and the intraluminal device 2102. The PIM 2304 can be communicatively positioned between the processing system 2306 and the intraluminal device 2102. In some embodiments, the PIM 2304 performs preliminary processing of data prior to relaying the data to the processing system 2306. In examples of such embodiments, the PIM 2304 performs amplification, filtering, and/or aggregating of the data. In an embodiment, the PIM 2304 also supplies high- and low-voltage DC power to support operation of the intraluminal device 2102 via the conductive members 2230.

A multi-filar cable or transmission line bundle 2230 can include a plurality of conductors, including one, two, three, four, five, six, seven, or more conductors. In the example shown in FIG. 15, the multi-filar conductor bundle 2230 includes two straight portions 2232 and 2236, where the multi-filar conductor bundle 2230 lies parallel to a longitudinal axis of the flexible elongate member 2106, and a spiral portion 2234, where the multi-filar conductor bundle 2230 is wrapped around the exterior of the flexible elongate member 2106 and then overcoated with an insulative and/or protective polymer 2240. Communication, if any, along the multi-filar conductor bundle 2230 may be through numerous methods or protocols, including analog, digital, serial, parallel, and otherwise, wherein one or more filars of the bundle 2230 carry signals. One or more filars of the multi-filar conductor bundle 2230 may also carry direct current (DC) power, alternating current (AC) power, or serve as a ground connection.

The display or monitor 2308 may be a display device such as a computer monitor or other type of screen. The display or monitor 2308 may be used to display selectable prompts, instructions, and visualizations of imaging data to a user. In some embodiments, the display 2308 may be used to provide a procedure-specific workflow to a user to complete an intraluminal imaging procedure.

It is noted that the intracranial sensing system 300 of FIG. 3 may be similar in some ways to the intraluminal sensing system 2100 of FIG. 15, though with some significant differences. For example, while the intraluminal sensing system 2100 is intended primarily for pressure measurements in a patient's blood vessels in a clinical (e.g., operating room) setting by highly skilled clinicians (e.g., a vascular surgical team), the intracranial sensing system 300 is intended primarily for pressure measurements of a patient's CSF, interior to the cranium, in a field or ambulance setting, by emergency medical personnel (e.g., a paramedic team). As such, in the intracranial sensing system 300, the display 2308 and processing system 2306 are combined into a single compact display monitor 310, which may or may not have any network communication capability. Additionally, in the intracranial sensing system 300 there is no equivalent to the PIM 2304 or connector 2314 of the intravascular sensing system 2100. Instead, the intracranial guidewire 102 connects directly into the display monitor 310. The intracranial guidewire 102 may also be significantly shorter than the intraluminal guidewire 2102, as it does not need to navigate the tortuous pathways of the human vascular system, and may contain only a single core wire 210 rather than a distal core wire 2210 and proximal core wire 2220 joined by a hypotube. In addition, the intracranial guidewire 102 may be more flexible than the intraluminal guidewire 2102, to reduce the chances of injuring the brain as the intracranial guidewire 102 is inserted into the cranium. Other differences may exist instead of or in addition to those described herein, to enhance the utility of the intracranial sensing system 300 for its intended function of safely and accurately measuring intracranial pressure in a field or ambulance setting.

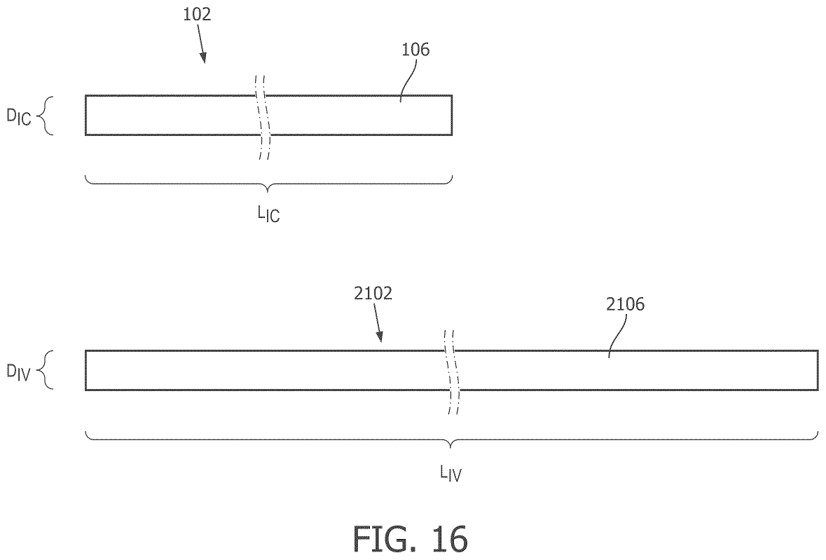

FIG. 16 is a schematic, diagrammatic size comparison between an intracranial pressure-sensing guidewire 102 and an intraluminal or intravascular pressure-sensing guidewire 2102, according to aspects of the present disclosure. Generally speaking, the functional requirements of an intracranial flexible elongate member 106 can be met with a shorter length than can the functional requirements of an intraluminal or intravascular flexible elongate member 106. Thus, while an intravascular pressure-sensing guidewire 2102 may have a length Liv of between 150 centimeters and 300 centimeters, for example, an intracranial pressure-sensing guidewire 102 may have a length L_{IC} of only 30 to 90 centimeters. The relatively much longer length of the intravascular guidewire is needed to reach a blood vessel in an interior of the body (heart, abdomen, etc.), through vasculature inside the body, from an access location at an external surface of the body (e.g., groin, arm, neck). The relatively much longer length of the intravascular guidewire can be cumbersome for a physician to handle/maneuver, but it is used in a controlled environment, such as a catheterization laboratory. The length of the intracranial guidewire can advantageously be much shorter than intravascular guidewire. The access location for the intracranial guidewire is an opening in the skull, and the measurement location (e.g., the subarachnoid space) is just inside the skull, so the intracranial guidewire only needs to extend for a relatively much shorter distance. Also, the intracranial guidewire can be used in emergency, uncontrolled situation (e.g., in the field), where a relatively much shorter length is easier for a user (e.g., a paramedic) to handle/maneuver. For both the intravascular pressure-sensing guidewire 2102 and the intracranial pressure-sensing guidewire 102, it may be desirable to have a small diameter (e.g., the smallest diameter that can reasonably be achieved while providing the functionality described herein). Thus the diameter D_{IC} of the intracranial pressure-sending guidewire 102 and the diameter D_{IV} of the intravascular pressure-sensing guidewire 2102 may both be in the range of 0.014 to 0.035 inches (360 to 890 microns).

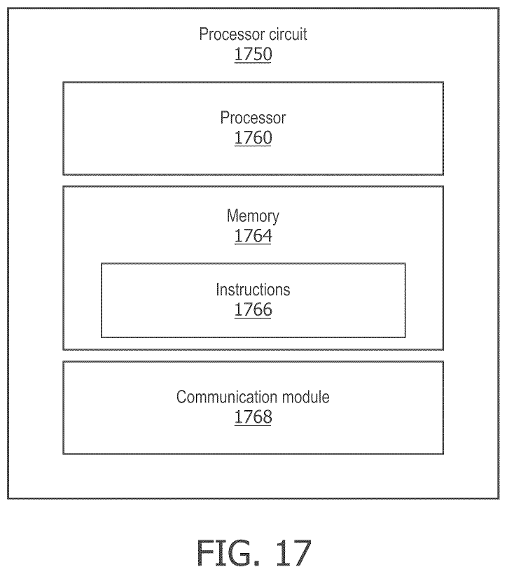

FIG. 17 is a schematic diagram of a processor circuit 1750, according to aspects of the present disclosure. The processor circuit 1750 may be implemented in the intracranial sensing system 300, the intraluminal sensing system 2100, the display monitor 310, the processing system 2306, or other devices or workstations (e.g., third-party workstations, network routers, etc.), or on a cloud processor or other remote processing unit, as necessary to implement the method. As shown, the processor circuit 1750 may include a processor 1760, a memory 1764, and a communication module 1768. These elements may be in direct or indirect communication with each other, for example via one or more buses.

The processor 1760 may include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 1760 may also comprise another hardware device, a firmware device, or any combination thereof configured to perform the operations described herein. The processor 1760 may also be implemented as a combination of computing devices, e.g., a combination of a DSP and a microprocessor, a plurality of microprocessors, one or more microprocessors in conjunction with a DSP core, or any other such configuration.

The memory 1764 may include a cache memory (e.g., a cache memory of the processor 1760), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 1764 includes a non-transitory computer-readable medium. The memory 1764 may store instructions 1766. The instructions 1766 may include instructions that, when executed by the processor 1760, cause the processor 1760 to perform the operations described herein. Instructions 1766 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 1768 can include any electronic circuitry and/or logic circuitry to facilitate direct or indirect communication of data between the processor circuit 1750, and other processors or devices. In that regard, the communication module 1768 can be an input/output (I/O) device. In some instances, the communication module 1768 facilitates direct or indirect communication between various elements of the processor circuit 1750 and/or the systems 300 or 2100. The communication module 1768 may communicate within the processor circuit 1750 through numerous methods or protocols. Serial communication protocols may include but are not limited to United States Serial Protocol Interface (US SPI), Inter-Integrated Circuit (I2C), Recommended Standard 232 (RS-232), RS-485, Controller Area Network (CAN), Ethernet, Aeronautical Radio, Incorporated 429 (ARINC 429), MODBUS, Military Standard 1553 (MIL-STD-1553), or any other suitable method or protocol. Parallel protocols include but are not limited to Industry Standard Architecture (ISA), Advanced Technology Attachment (ATA), Small Computer System Interface (SCSI), Peripheral Component Interconnect (PCI), Institute of Electrical and Electronics Engineers 488 (IEEE-488), IEEE-1284, and other suitable protocols. Where appropriate, serial and parallel communications may be bridged by a Universal Asynchronous Receiver Transmitter (UART), Universal Synchronous Receiver Transmitter (USART), or other appropriate subsystem.

External communication (including but not limited to software updates, firmware updates, preset sharing between the processor and central server, or readings from the pressure sensor) may be accomplished using any suitable wireless or wired communication technology, such as a cable interface such as a universal serial bus (USB), micro USB, Lightning, or FireWire interface, Bluetooth, Wi-Fi, ZigBee, Li-Fi, or cellular data connections such as 2G/GSM (global system for mobiles), 3G/UMTS (universal mobile telecommunications system), 4G, long term evolution (LTE), WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. Information may also be transferred on physical media such as a USB flash drive or memory stick.

As will be readily appreciated by those having ordinary skill in the art after becoming familiar with the teachings herein, the intracranial pressure sensing device advantageously provides an accurate, minimally invasive tool for measuring intracranial pressure in field and ambulance settings, to provide earlier warnings for head-trauma patients in need of treatment for high intracranial pressure.

A number of variations are possible on the examples and embodiments described above. For example, the intracranial guidewire may be longer, shorter, thicker, or thinner than described herein. Entry points for the guidewire to the CSF may be other than those described herein. The sensor may include an imaging sensor, temperature sensor, flow sensor, color sensor, proximity sensor, immersion sensor, or other sensing modalities. In some cases, the intracranial pressure-sensing guidewire can be used to guide the insertion of other devices into the skull, such as subdural catheters, needles, or hypotubes, for removal of CSF from the cranium (e.g., to relieve pressure) and/or injection of medications or other materials into the CSF or other layers of the cranium (e.g., to reduce swelling or effect surgical repair or intervention).

The logical operations making up the embodiments of the technology described herein are referred to variously as operations, steps, objects, elements, components, or modules. It should be understood that these may occur or be performed or arranged in any order, unless explicitly claimed otherwise or a specific order is inherently necessitated by the claim language.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the intracranial pressure sensing device. Connection references, e.g., attached, coupled, connected, joined, or “in communication with” are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary embodiments of the intracranial pressure sensing device as defined in the claims. Although various embodiments of the claimed subject matter have been described above with a certain degree of particularity, or with reference to one or more individual embodiments, those skilled in the art could make numerous alterations to the disclosed embodiments without departing from the spirit or scope of the claimed subject matter.

Still other embodiments are contemplated. It is intended that all matter contained in the above description and shown in the accompanying drawings shall be interpreted as illustrative only of particular embodiments and not limiting. Changes in detail or structure may be made without departing from the basic elements of the subject matter as defined in the following claims.

What is claimed is:

1. An apparatus, comprising:
an intracranial guidewire comprising:
a flexible elongate member configured for insertion into a subarachnoid space through a skull of a patient; and
a pressure sensor disposed at a distal portion of the flexible elongate member and configured to measure a pressure of cerebrospinal fluid inside of the subarachnoid space.

2. The apparatus of claim 1, wherein the flexible elongate member comprises a core wire configured to provide structural support for the intracranial guidewire.

3. The apparatus of claim 1, wherein the flexible elongate member does not include a lumen.

4. The apparatus of claim 1, wherein a length of the flexible elongate member is between 30 cm and 90 cm.

5. The apparatus of claim 1, further comprising:
a plurality of electrical conductors extending along a length of the flexible elongate member and configured to carry electrical signals associated with the pressure sensor; and
a connection portion disposed at a proximal portion of the flexible elongate member and in electrical communication with the pressure sensor via the plurality of electrical conductors.

6. A system for measuring intracranial pressure, the system comprising:
an intracranial guidewire configured to be inserted through an opening in a skull of a patient, wherein the intracranial guidewire comprises:
a pressure sensor disposed at a distal portion of the intracranial guidewire and configured to generate signals representative of a pressure of cerebrospinal fluid inside the skull; and
a connection portion disposed at a proximal portion of the intracranial guidewire and in communication with the pressure sensor; and
a display monitor comprising:
a housing;
a connection port coupled to the housing, wherein the connection port is configured to receive the proximal portion of the intracranial guidewire and establish communication with the pressure sensor via the connection portion;
a processor circuit disposed inside the housing and configured to receive the signals generated by the pressure sensor via the connection port and connection portion of the intracranial guidewire; and
a display coupled to the housing and in communication with the processor circuit, wherein the display is configured to output a visual representation of the pressure of the cerebrospinal fluid based on the signals generated by the pressure sensor.

7. The system of claim 6, wherein the display monitor further comprises:
a power supply disposed inside the housing and configured to provide power to at least one of the processor circuit, the display, or the connection port.

8. The system of claim 6,
wherein the display monitor further comprises a user control coupled to the housing and in communication with the processor circuit,
wherein an actuation of the user control by a user is configured to perform at least one of:
provide power to the pressure sensor;
calibrate the pressure sensor; or
trigger the display of the visual representation of the pressure of the cerebrospinal fluid.

9. The system of claim 6,
further comprising an access drill configured to create a burr hole in the skull,
wherein the opening comprises the burr hole.

10. The system of claim 6,
further comprising a channel plug configured to be positioned within the opening,
wherein the channel plug comprises a lumen extending completely through the channel plug and configured to receive the intracranial guidewire during the insertion of the intracranial guidewire through the opening.

11. The system of claim 6, wherein the visual representation of the pressure of the cerebrospinal fluid comprises a numerical value.

12. The system of claim 6, wherein the processor is configured to:
determine the pressure of the cerebrospinal fluid based on the signals generated by the pressure sensor;
compare the pressure of the cerebrospinal fluid to a threshold value;
determine if the pressure of the cerebrospinal fluid exceeds the threshold value based on the comparison; and
activate a threshold alarm in response to the determination that the pressure of the cerebrospinal fluid exceeds the threshold value.

13. The system of 12, wherein the threshold alarm comprises a further visual representation on the display.

14. The system of claim 12,
wherein the display monitor further comprises a speaker disposed inside the housing and in communication with the processor circuit,
wherein the threshold alarm comprises an audible alarm output by the speaker.

15. The system of claim 12,
wherein the display monitor further comprises an indicator light coupled to the housing and in communication with the processor circuit,
wherein the threshold alarm comprises activation of the indicator light.
